(19) United States
(12) Patent Application Publication
(10) Pub. No.: US 2024/0122330 A1
(43) Pub. Date: Apr. 18, 2024

(54) METHOD AND APPARATUS FOR NAIL FUNGUS TREATMENT

(71) Applicant: Daniel O. Sokoloff, Jupiter, FL (US)

(72) Inventor: Daniel O. Sokoloff, Jupiter, FL (US)

(21) Appl. No.: 17/968,761

(22) Filed: Oct. 18, 2022

(51) Int. Cl. A45D 29/17

(52) U.S. Cl. CPC A45D 29/17

(57) ABSTRACT

A method for treating nail fungus comprises providing a treatment element including a flexible elongate body. The elongate body has a first end and a second end opposite the first end. The first and second ends define a length therebetween. An outer surface is defined along the length between the first and second ends. The method further includes advancing at least a portion of the length of the treatment element to a target treatment site underneath a nail of a user. The target treatment site is an area of fungal debris build-up underneath the nail. The method further includes dislodging at least a portion of the fungal debris build-up and simultaneously applying a therapeutic agent to the target treatment area. The treatment element is then retracted from underneath the nail and extracts at least a portion of the dislodged fungal debris build-up.

### CROSS-REFERENCE TO RELATED APPLICATION

n/a

### FIELD

The present invention relates to the field of nail fungus treatment, and in particular, apparatuses and methods of uses thereof, for treating nail fungus.

### BACKGROUND

Toenail fungus is prevalent on many walking surfaces and other surfaces to which the toe nail can be exposed. Onychomycosis is a fungal infection of the nail, which occurs frequently due to the prevalence of toenail fungus. Onychomycosis may lead to symptoms such as foul odor, discoloration and hyperkeratosis or onychauxis—the subungual thickening of the nail— which may eventually result in the nail becoming brittle and eventually onycholysis—the separation of the nail from the nail bed. Onycholysis may occur because when fungus gets under toenail, the fungus metabolizes or eats the toenail and causes subungual keratin to build up under the nail. As the subungual keratin accumulates under the nail, the subungual keratin build-up lifts the nail and separates the nail from the nail bed.

Various treatments for toenail fungus involve applying topical medicine creams and ointments on top of the nail and at the outer end of the nail. However, the application of topical medicine on the top and end of the nail does not fully penetrate into the fungus or the subungual keratin build-up underneath the nail.

The subungual keratin build-up can also be excavated from underneath the nail with a pick-type instrument. The pick-type instruments only allow for axial cleaning in that the pick is only able to clean the small portion of the nail that the pointed end of the pick touches. Thus, due to the axial cleaning motion, the pick-type instrument must be moved back and forth many times before the entire width of the underside of the nail can be cleaned. The axial insertion of the pick can also push and compact the debris from the subungual keratin build-up to the rear of the nail making removal of the debris more difficult.

### SUMMARY OF THE INVENTION

Embodiments of the present invention address deficiencies of the art in respect to nail fungus treatment and provide a novel and non-obvious method and apparatus for nail fungus treatment.

According to one or more embodiments, a method for treating nail fungus comprises providing a treatment element including a flexible elongate body. The elongate body has a first end and a second end opposite the first end. The first and second ends define a length therebetween. An outer surface is defined along the length between the first and second ends. The method further includes advancing at least a portion of the length of the treatment element to a target treatment site underneath a nail of a user. The target treatment site is an area of fungal debris build-up underneath the nail. The method further includes dislodging at least a portion of the fungal debris build-up and simultaneously applying a therapeutic agent to the target treatment area. The treatment element is then retracted from underneath the nail and extracts at least a portion of the dislodged fungal debris build-up.

In one aspect, at least one of planar movement and lateral movement is applied to the treatment element when dislodging at least a portion of the fungal debris build-up and applying the therapeutic agent.

In another aspect, at least a portion of the outer surface is coated with the therapeutic agent.

In another aspect, the elongate body is saturated with a therapeutic agent.

In another aspect, the nail is disposed between the first and second ends of the elongate body when the treatment element is advanced towards the target treatment site.

In another aspect, the treatment element is undulated in profile and has a plurality of protrusions defined along the length.

In another aspect, the treatment element is one of a string, a thread, a filament, a bundle of filaments, a wire, a cable, and a thin tape. The treatment element has a flexibility such that the elongate body is capable of being contorted and manipulated to achieve a desired shape to follow up the contours of the target site under the nail.

In another aspect, first and second ends of the elongate body are each configured to wrap around a user's fingers prior to and during movement of the treatment element underneath the nail.

In another aspect, the first and second ends of the elongate body are each coupled to a handle of a treatment device sized and configured to be held by a user.

In another aspect, the treatment element is formed from at least one material selected from the group consisting of nylon, Teflon, polypropylene, silk, polyester, or any material, or any combination thereof.

In another aspect, the elongate body is formed of a plurality of filaments wound together in a bundle.

According to one or more additional embodiments, a nail fungus treatment apparatus comprises a treatment element including a flexible elongate body that defines a length and has an outer surface. The outer surface is coated in a therapeutic agent. The elongate body is sized and configured to be received underneath a nail of a user and advanced towards a target treatment site. The target treatment site is an area of fungal debris buildup underneath the nail.

In one aspect, the treatment element is configured to dislodge and extract at least a portion of fungal debris buildup through at least one of planar movement and lateral movement of the elongate body outwardly towards a distal edge of the nail; and simultaneously apply the therapeutic agent to the target treatment site when in contact with the target treatment site.

In another aspect, the treatment element is saturated with the therapeutic agent.

In another aspect, the elongate body is undulated in profile and has a plurality of protrusions defined along the length.

In another aspect, the treatment element is one of a string, a thread, a filament, a bundle of filaments, a wire, a cable, and a thin tape.

In another aspect, the elongate body includes a first end and a second end opposite the first end, the first and second ends of the elongate body are each configured to wrap around a user's fingers prior to and during movement of the treatment element underneath the nail.

In another aspect, the elongate body includes a first end and an opposite second end each coupled to a handle of a treatment device sized and configured to be held by a user.

In another aspect, the treatment element is formed from at least one material selected from the group consisting of nylon, Teflon, polypropylene, silk, polyester, or any material, or any combination thereof.

According to one or more embodiments, a nail fungus treatment apparatus comprises a treatment element including a flexible elongate body. The flexible elongate body has a first end and a second end opposite the first end. The first and second ends define a length therebetween. The flexible elongate body further has an outer surface defined along the length between the first and second ends, the outer surface is one of coated with or saturated with an antifungal medication. The treatment element is sized and configured to: be received underneath a nail of a user and advanced towards a target treatment site, the target treatment site being an area of fungal debris buildup underneath the nail; dislodge and extract at least a portion of fungal debris buildup through at least one of planar movement and lateral movement of the flexible elongate body towards a distal edge of the nail; and when in physical contact with the target treatment site, apply the therapeutic agent to the target treatment site.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 illustrates the treatment of a target nail with a flexible treatment element in accordance with the principles of the present invention;

FIG. 2 illustrates the flexible treatment element of FIG. 1 constructed in accordance with the principles of the present invention;

FIG. 3 illustrates the flexible treatment element of FIGS. 1-2 defining a plurality of protrusions in accordance with the principles of the present invention;

FIG. 4A illustrates the positioning of the treatment element of FIGS. 1-3 prior to advancement underneath a target nail of a patient, in accordance with the principles of the present invention;

FIG. 4B illustrates the positioning of the treatment element of FIGS. 1-4A when advanced to a target treatment site underneath the target nail, in accordance with the principles of the present invention;

FIGS. 4C and 4D illustrate lateral movement of the treatment element of FIGS. 1-4B when positioned at the target treatment site in order to dislodge fungal debris, in accordance with the principles of the present invention;

FIG. 4E illustrates the extraction of fungal debris from the target treatment site once the treatment element of FIGS. 1-4D is retracted from underneath the target nail, in accordance with the principles of the present invention;

FIG. 5 illustrates an exemplary embodiment in which the treatment element of FIGS. 1-4E is affixed to a distal end of a handle, in accordance with the principles of the present invention; and

FIG. 6 illustrates a flowchart of process of treating nail fungus with the treatment element of FIGS. 1-5, in accordance with the principles of the present invention.

### DETAILED DESCRIPTION OF PREFERRED EMBODIMENTS

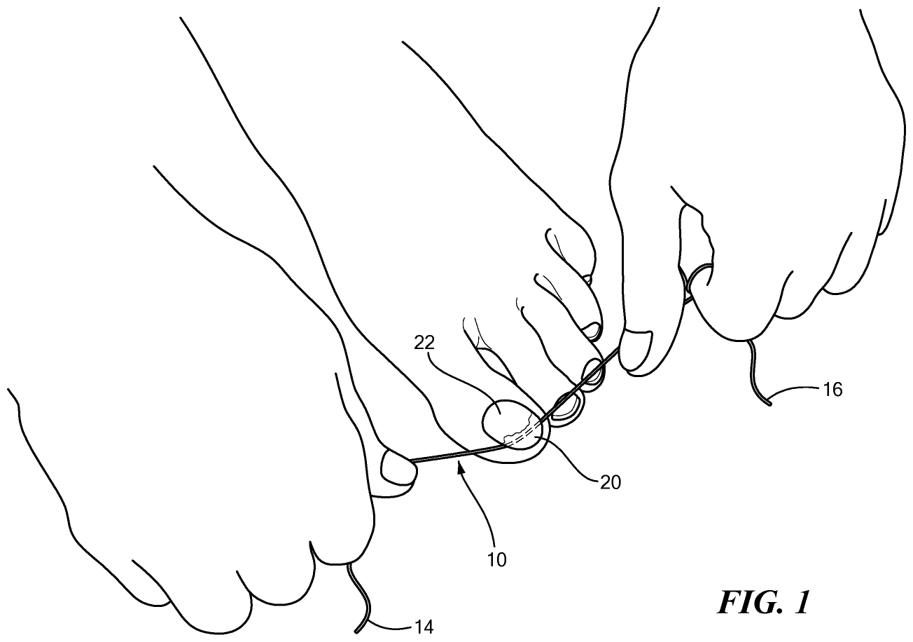

Embodiments of the invention provide for a method and apparatus for nail fungus treatment. Referring now to the drawing figures in which like reference designators refer to like elements, there is shown in FIG. 1 an exemplary apparatus suitable for treating nail fungus. In one or more embodiments, the apparatus includes a treatment element designated generally as “10,” and which has a flexible elongate body 12. The flexibility of the elongate body 12 allows the treatment element 10 to have a free-form body that is capable of freely contorting and stretching to achieve various positions, and also allows the treatment element 10 to be freely manipulated by the user to achieve a desired shape or other positioning, particularly to substantially match the contours under the user's, which can vary among users but can change during the treatment process as debris is removed. The elongate body 12 has a first end 14, a second end 16 opposite the first end 14, and defines a length therebetween. An outer surface 18 is defined along the length between the first and second ends 14, 16. The elongate body 12 of the treatment element 10 may have an undulated profile and circular or substantially round cross-section that is smaller than its length. The treatment element 10 may be a flexible string, thread, filament, bundle of filaments wound together in a bundle, wire, cable, or a thin-tape sized and configured to be received under the free edge 20 and nail plate 22 of a user, subject, or patient's toenail or fingernail, and is coated or saturated with antifungal medicine. As a non-limiting example, the treatment element 10 may be formed at least one of nylon, Teflon, polypropylene, silk, polyester, or any material, or any combination thereof.

It is to be understood that as used herein, “string” may be used to refer generally to a flexible and manipulatable thread, filament, bundle of filaments, wire, cable, or thin tape sized and configured to be received under the free edge 20 and nail plate 22 of the user. The flexible elongate body can be regarded as thin or slender having a relatively small cross section, for example on the order of 50-100 mils in diameter, relative to the length of the elongate body.

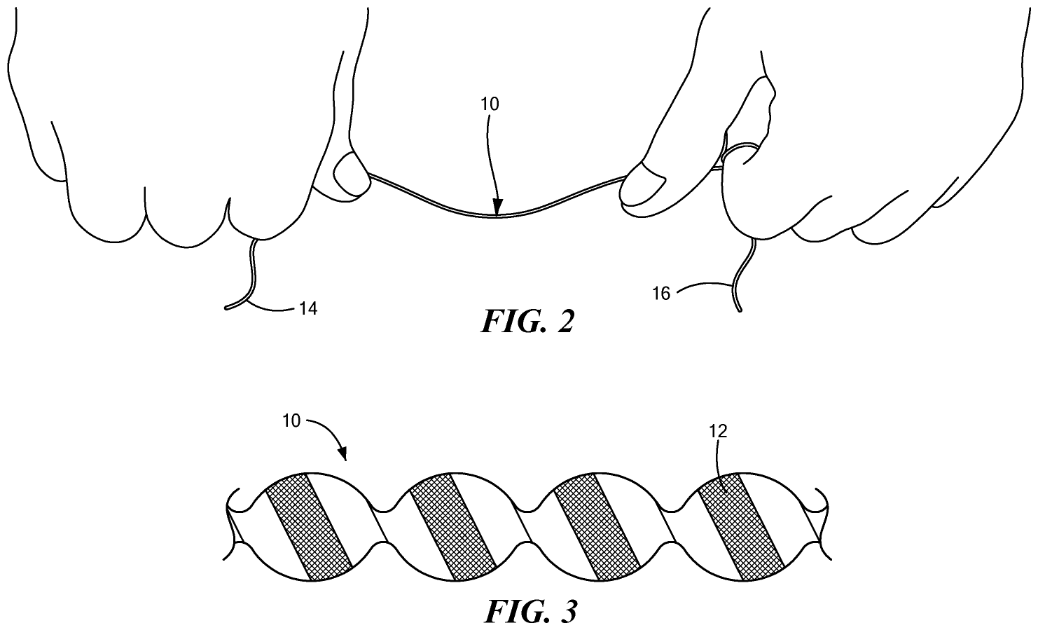

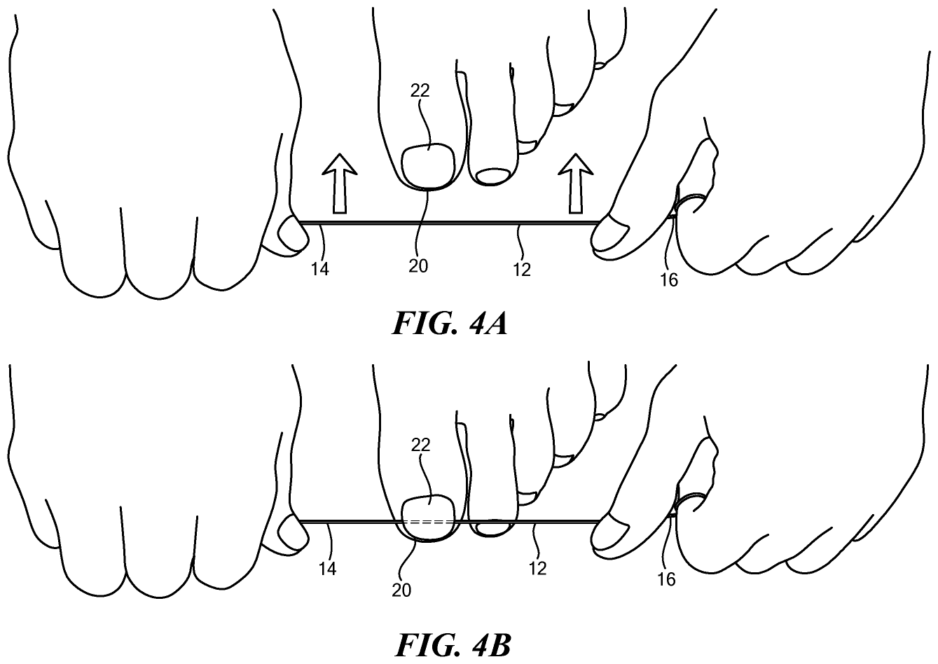

As shown in FIG. 2, the treatment element 10 may be coated or saturated with a therapeutic agent suitable for treating, reducing the growth and spread of, or eliminating subungual keratin build-up (i.e., fungal debris) underneath the nail—such as an antifungal medicine. The antifungal medicine may be any medicine used to treat nail infections such as, for example, terbinafine, ketoconazole, clotrimazole, zinc undecylenate, undecylenic acid, etc., or any antifungal medication, or any combination thereof. Additionally, in some embodiments the treatment element 10 may have an undulated profile and uniform shape or diameter along the length of the elongate body 12. However, in other embodiments, such as shown those in FIG. 3, the treatment element 10 may have a spiral or braided configuration that increases and decreases in diameter along the length of the elongate body 12. In some such configurations, the elongate body 12 may define protrusions along the length that are configured to facilitate the scraping of subungual keratin build-up from underneath the nail when in contact with the target treatment site.

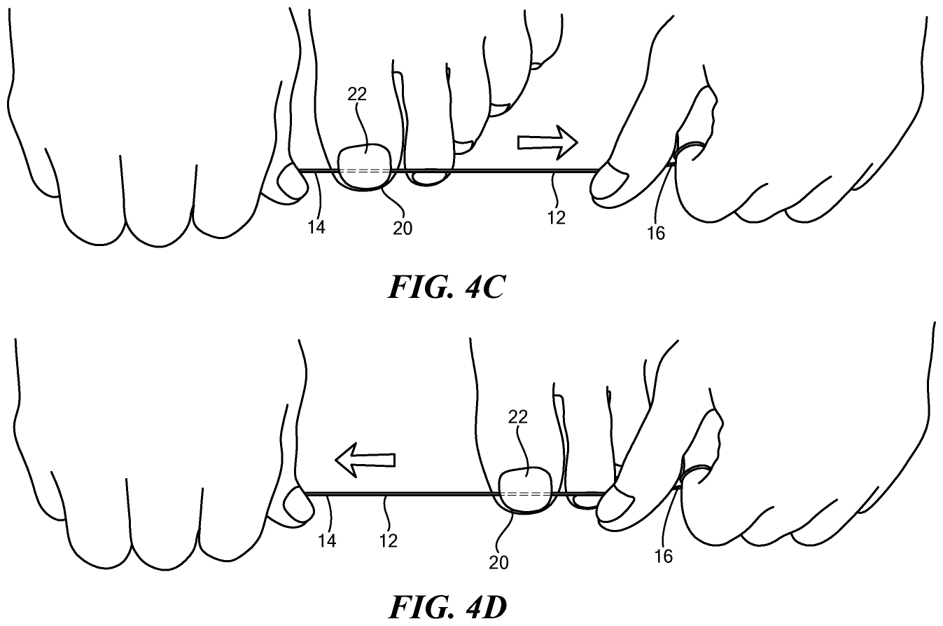

Now referring to FIGS. 4A-4E, use of the treatment element 10 during a treatment procedure is shown. As shown, the first and second ends 14, 16 of the elongate body 12 may be held by a user. For example, the first end 14 may be grasped by the user's left hand and the second end may be grasped by the user's right hand. As shown in FIG. 4A, the treatment element 10 is then extended underneath the free edge 20 of the nail (toenail or fingernail) and the nail plate 22, and advanced towards a target treatment site within the nail bed. As described herein, the target treatment site is an area of subungual keratin build-up underneath the nail plate 22. The treatment element 10 is advanced from the distal end of the free edge 20 towards the back of the nail bed or proximal end of the nail edge that ends at the nail bed (FIG. 4B), and is moved without scraping the subungual keratin build-up in order to avoid compacting and pushing debris further underneath the nail plate 22. Upward pressured is then applied to the subungual keratin build-up through treatment element 10 and the treatment element is then retracted from the rear of the nail towards the free edge 20 to scrape the subungual keratin build-up and apply the antifungal medicine simultaneously. The treatment element 10 may also be moved side-to-side, such as in FIGS. 4C-4D, underneath the nail to remove additional subungual keratin build-up and apply the antifungal medicine. Once the treatment element 10 has scraped a desired amount of subungual keratin build-up, it may then be retracted and removed from underneath the user's nail (FIG. 4E).

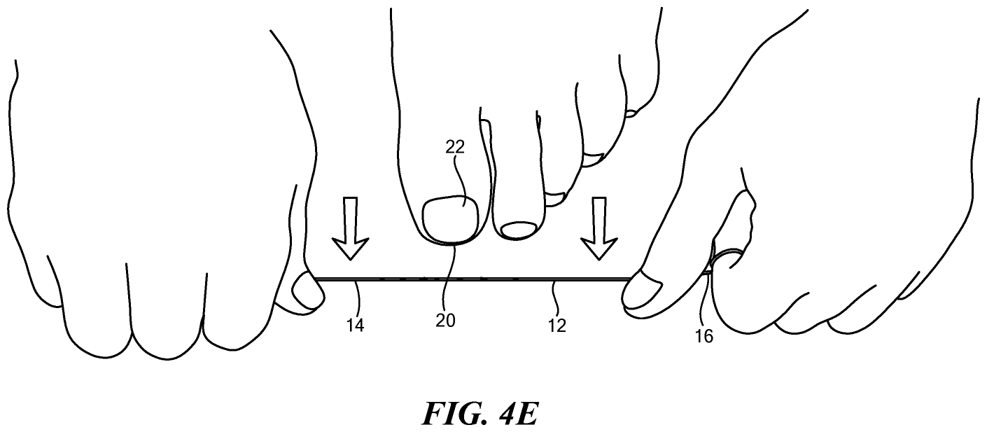

The outward movement of the treatment element 10 allows for a planar cleaning motion— as opposed to an axial cleaning motion—of the underside of the nail as the treatment element 10 is able to extend across the entire width (side-to-side) of the nail or at least a larger portion of the width of the nail than axial cleaning motion would allow. The treatment element 10 may be moved outwardly in a planar cleaning motion substantially perpendicular to the sides of the nail to extend across the entire width (side-to-side) of the nail. Additionally, although though not shown, a user may also extend the treatment element 10 less than the entire width of the nail to allow a planar cleaning motion of the underside of the nail at different angles to further remove subungual keratin build-up and apply antifungal medicine. The string-like apparatus may be wrapped around the end-user's fingers or may be attached to a handle or frame (as shown in FIG. 5) that allows the end user to hold the frame in between the fingers. An undulated profile may provide protrusions to allow the string-like apparatus to scrape subungual keratin build-up from underneath the nail.

According to one or more embodiments, the therapeutic agent of the treatment element 10 may also be topically applied to a top or outer surface of the user's nail plate 22. For example, rather than advancing and maneuvering the treatment element 10 under the user's nail plate 22, the treatment element 10 may instead be used or brushed against the top or outer surface of the nail plate 22 to apply the therapeutic agent directly to the top or outer surface of the nail plate 22. This allows users to apply the therapeutic agent to both the top or outer surface of their nail plate 22, as well as underneath the nail plate 22, to more effectively treat or inhibit nail fungus growth. As a non-limiting example, when applied topically to the nail plate 22, the treatment element 10 may be the flexible “string”-like apparatus.

Now referring to FIG. 5, the treatment element 10 may be attached, affixed, or otherwise coupled to a distal end 24 of a handle 26 that may be held between an end user's fingers to assist the user in reaching the targeted nail for treatment and/or advancing the treatment element 10 in the planar cleaning motion underneath the nail. However, it is to be understood, that in some embodiments, the treatment element 10 is not coupled to a handle 26, and thus, may be wrapped around an end user's fingers to extend the string-like apparatus in the planar cleaning motion underneath the nail.

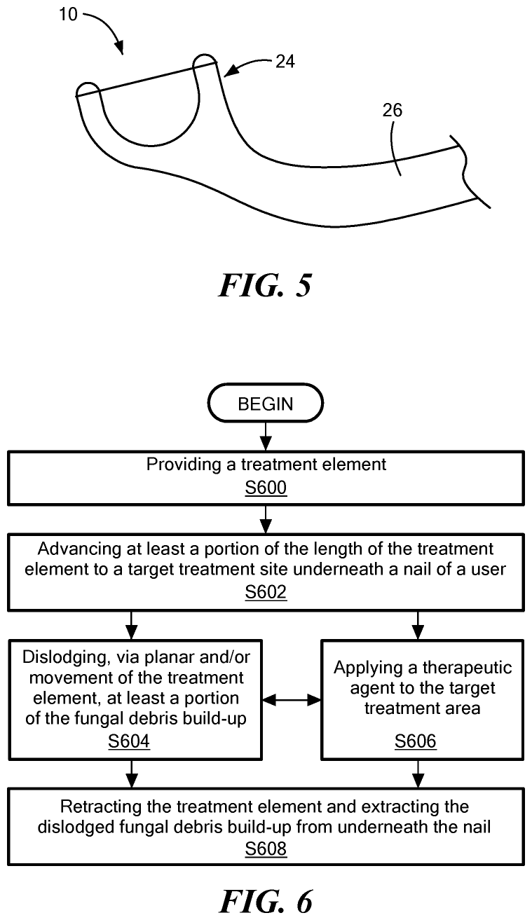

Now referring to FIG. 6, a flowchart illustrating a method of treating nail fungus is shown. The method includes providing the treatment element 10 (whether physically grasped by a user's hands, wrapped around the user's fingers, or coupled to the distal end 24 of the handle 26) (Block S600) and advancing at least a portion of the length of the treatment element to the target treatment site underneath a nail of a user, subject, or patient (Block S602). The target treatment site is the area of subungual keratin build-up underneath the nail. The treatment element 10 then applies force to the fungal debris in a planar and/or lateral motion to scrape and/or dislodge the fungal debris from the nail bed (Block S604). As the treatment element 10 is in contact with the fungal debris, the antifungal medicine that is coated on or saturated in the treatment element 10 is simultaneously applied to the target treatment site (including the nail bed) to reduce or eliminate subungual keratin build-up (Block S606). Once a desired amount of fungal debris has been dislodged, the fungal debris is then extracted from underneath the nail by retracting the treatment element 10 outwardly past the free edge 20 of the nail plate 22 (Block S608).

Although specific embodiments of the invention have been disclosed, those having ordinary skill in the art will understand that changes can be made to the specific embodiments without departing from the spirit and scope of the invention. The scope of the invention is not to be restricted, therefore, to the specific embodiments. Furthermore, it is intended that the appended claims cover any and all such applications, modifications, and embodiments within the scope of the present invention.

Finally, the terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “includes” and/or “including,” when used in this specification, specify the presence of stated features, integers, steps, operations, elements, and/or components, but do not preclude the presence or addition of one or more other features, integers, steps, operations, elements, components, and/or groups thereof.

The corresponding structures, materials, acts, and equivalents of all means or step plus function elements in the claims below are intended to include any structure, material, or act for performing the function in combination with other claimed elements as specifically claimed. The description of the present invention has been presented for purposes of illustration and description, but is not intended to be exhaustive or limited to the invention in the form disclosed. Many modifications and variations will be apparent to those of ordinary skill in the art without departing from the scope and spirit of the invention. The embodiment was chosen and described in order to best explain the principles of the invention and the practical application, and to enable others of ordinary skill in the art to understand the invention for various embodiments with various modifications as are suited to the particular use contemplated.

Having thus described the invention of the present application in detail and by reference to embodiments thereof, it will be apparent that modifications and variations are possible without departing from the scope of the invention defined in the appended claims as follows:

What is claimed is:

1. A method for treating nail fungus, comprising:
providing a treatment element, the treatment element including a flexible elongate body, whereby the elongate body is capable of being contorted and manipulated to achieve a desired shape beneath the nail, and having:
a first end and a second end opposite the first end, the first and second ends defining a length therebetween;
an outer surface defined along the length between the first and second ends; and
presenting a therapeutic agent on the outer surface; and
advancing at least a portion of the length of the treatment element to a target treatment site underneath a nail of a user, the target treatment site being an area of fungal debris build-up underneath the nail;
dislodging at least a portion of the fungal debris build-up and simultaneously applying the therapeutic agent to the target treatment area; and
retracting the treatment element from underneath the nail and extracting at least a portion of the dislodged fungal debris build-up.

2. The method of claim 1, wherein at least one of planar movement and lateral movement is applied to the treatment element when dislodging at least a portion of the fungal debris build-up and applying the therapeutic agent.

3. The method of claim 1, wherein at least a portion of the outer surface is coated with the therapeutic agent.

4. The method of claim 1, wherein the elongate body is saturated with a therapeutic agent.

5. The method of claim 1, wherein the nail is disposed between the first and second ends of the elongate body when the treatment element is advanced towards the target treatment site.

6. The method of claim 1, wherein the elongate body is undulated in profile and has a plurality of protrusions defined along the length.

7. The method of claim 1, wherein:
the elongate body is one of a string, a thread, a filament, a bundle of filaments, a wire, a cable, and a thin tape.

8. The method of claim 1, wherein the first and second ends of the elongate body are each configured to wrap around a user's fingers prior to and during movement of the treatment element underneath the nail.

9. The method of claim 1, wherein the first and second ends of the elongate body are each coupled to a handle of a treatment device sized and configured to be held by a user.

10. The method of claim 1, wherein the treatment element is formed from at least one material selected from the group consisting of nylon, Teflon, polypropylene, silk, polyester, or any material, or any combination thereof.

11. The method of claim 1, wherein the elongate body is formed of a plurality of filaments wound together in a bundle.

12. A nail fungus treatment apparatus, comprising:
a treatment element including a flexible elongate body defining a length and having an outer surface, and presenting a therapeutic agent on the outer surface, the elongate body having a flexibility such that the elongate body is capable of being contorted and manipulated to achieve a desired shape;
the elongate body being sized and configured to be received underneath a nail of a user and advanced towards a target treatment site; and
wherein the target treatment site is an area of fungal debris buildup underneath the nail.

13. The apparatus of claim 12, wherein the treatment element is configured to:
dislodge and extract at least a portion of fungal debris buildup through at least one of planar movement and lateral movement of the elongate body outwardly towards a distal edge of the nail; and
simultaneously apply the therapeutic agent to the target treatment site when in contact with the target treatment site.

14. The apparatus of claim 12, wherein the treatment element is saturated with the therapeutic agent.

15. The apparatus of claim 12, wherein the elongate body is undulated in profile and has a plurality of protrusions defined along the length.

16. The apparatus of claim 12, wherein the treatment element is one of a string, a thread, a filament, a bundle of filaments, a wire, a cable, and a thin tape.

17. The apparatus of claim 12, wherein the elongate body includes a first end and a second end opposite the first end, the first and second ends of the elongate body are each configured to wrap around a user's fingers prior to and during movement of the treatment element underneath the nail.

18. The apparatus of claim 12, wherein the elongate body includes a first end and an opposite second end each coupled to a handle of a treatment device sized and configured to be held by a user.

19. The apparatus of claim 12, wherein the treatment element is formed from at least one material selected from the group consisting of nylon, Teflon, polypropylene, silk, polyester, or any material, or any combination thereof.

20. A nail fungus treatment apparatus, comprising:
a treatment element including a flexible elongate body having:
a first end and a second end opposite the first end, the first and second ends defining a length therebetween; and
an outer surface defined along the length between the first and second ends, the outer surface presenting an antifungal medication; and
wherein the treatment element is sized and configured to:
be received underneath a nail of a user and advanced towards a target treatment site, the target treatment site being an area of fungal debris buildup underneath the nail;
dislodge and extract at least a portion of fungal debris buildup through at least one of planar movement and lateral movement of the flexible elongate body towards a distal edge of the nail; and
when in physical contact with the target treatment site, apply the therapeutic agent to the target treatment site.
